(19) United States
(12) Patent Application Publication
(10) Pub. No.: US 2022/0378657 A1
(43) Pub. Date: Dec. 1, 2022

(54) SYSTEMS AND COMPONENTS FOR DRUG DELIVERY AND COMPONENTS FOR PREPARATION OF THE SAME

(71) Applicant: AMGEN INC., Thousand Oaks, CA (US)

(72) Inventors: Scott R. Gibson, Thousand Oaks, CA (US); Olivia Li, Simi Valley, CA (US); Nicholas J. Clark, Thousand Oaks, CA (US); Adam B. McCullough, Westlake Village, CA (US)

(21) Appl. No.: 17/770,320

(22) PCT Filed: Oct. 22, 2020
(86) PCT No.: PCT/US20/56784

§ 371 Date: Apr. 20, 2022

Related U.S. Application Data
(60) Provisional application No. 62/925,636, filed on Oct. 24, 2019.
(60) Provisional application No. 62/925,618, filed on Oct. 24, 2019.

(51) Int. Cl. A61J 1/20; A61J 1/14

(52) U.S. Cl. CPC A61J 1/2096; A61J 1/2006; A61J 1/1406; A61J 1/2089

(57) ABSTRACT

A device for connecting tubes for a drug delivery system includes a connecting portion having a first tube channel, a second tube channel, a connecting heating element, and a carriage assembly. The first tube channel is adapted to receive a first tube, and the second tube channel is adapted to receive a second tube. The connecting heating element is positioned adjacent to and/or intersecting the first and second tube channels and is adapted to selectively heat at least a portion of the first and second tube. The carriage assembly moves the heated first and second tubes into end-to-end contact with each other to couple the first and second tubes together.

### CROSS-REFERENCE TO RELATED APPLICATIONS

The present application claims priority to United States Application Nos. 62/925,636, entitled “Device for Welding and Sealing Drug Delivery Tube,” filed Oct. 24, 2019, and 62/925,618, entitled “Systems and Components for Drug Delivery and Components for Preparation of the Same,” filed Oct. 24, 2019. The priority applications are hereby incorporated by reference in their entireties.

### FIELD OF DISCLOSURE

The present disclosure generally relates to drug delivery devices and components of drug delivery systems and components for preparing the same for use.

### BACKGROUND

Drugs are administered to treat a variety of conditions and diseases. Intravenous (“IV”) therapy is a drug dosing process that delivers drugs directly into a patient's vein using an infusion contained in a delivery container such as IV bag and tubing connected to a needle subsystem that fluidically communicates with a reservoir through the pump assembly collectively called an infusion set. These drug dosings may be performed in a healthcare facility, or in some instances, at remote locations such as a patient's home. In certain applications, a drug delivery process may last for an extended period of time (e.g., for one hour or longer) or may include continuous or semi-continuous delivery of a drug over an extended period of time (e.g., for several hours, days, weeks, or longer). For many of these relatively long-term delivery requirements, a pump is often utilized to control and/or administer the drug to the patient. The pump may be coupled (physically, fluidly, and/or otherwise) to various components, such as a drug delivery container, supply lines, connection ports, and/or the patient.

In some environments, a pump and/or portable system and/or wearable may be desired. It may also be desirable to utilize a pump and an overall system that minimizes patient inconvenience, minimizes the size and profile of the device and the overall system, minimizes the complexity of the device and overall system, minimizes the noise and vibration of the device, accommodates easy connection/disconnection and changeover of the infusion set, simplifies or automates priming of the line, accommodate easy delivery interruption and reestablishment based on required therapy and delivery profile, easily provides status of delivery and other important user information such as occlusion and volume of drug delivered or remaining in the reservoir, reduces the cost of the device and the overall system, increases the reliability and accuracy of the device and the overall system. A fluid path may be disconnected and/or connected by connectors and/or adaptors, but such components may reduce user flexibility by only permitting disconnection and/or connection at certain, predefined locations. Additionally, such connectors and/or adaptors may be expensive, confusing to use, or unavailable for a user.

Some infusion therapies, such as therapies for oncology patients, can last for extended durations (e.g., up to 24 hours a day for up to 7 consecutive days). During continuous infusion, patients may be tethered to their infusion pumps and the required tubing sets/connections to receive therapy. This may become limiting to the patient's basic day to day activities such as bathing and changing clothes. In addition, patients may desire the ability to disconnect from their therapies for at least a portion of the day, such as for a brief period of time, which may be allowable when using certain oncology products. Some commercial sterile connectors may be integrated as part of the infusion lines to allow the patients to disconnect and reconnect while still maintaining sterility with additional cleaning/swabbing requirements prior to each reconnection. In some such examples, it is recommended that the IV sets are changed every 24 hours. As such, integrating the sterile disconnector may be limiting to the patient's desire beyond the 24-hour timeframe. Further, such sterile environment studies are typically conducted in a clinical or laboratory setting which may provide optimal use conditions compared to that of a patient's home.

Further, when reconstituting these drugs for administration, it is of particular importance to maintain a sterile environment so as to not taint or otherwise damage the quality of the drug. Additionally, some classes of drugs such as Bi-specific T-cell engagers may require exceptionally accurate quantities of the drug product and/or other fluids required for dosing so as to prevent the drug product from becoming toxic. Oftentimes, the healthcare professional must prepare the drug by closely following a set of steps to ensure a sterile environment is maintained and that correct quantities of ingredients are added to the delivery container. When reconstituting these drugs for administration, it may be desirable or necessary to utilize a diluent, such as by adding a diluent to a drug product vial. As a result of these various steps and requirements, the reconstitution process may be time-consuming, tedious, and may have an unacceptable or undesirable error rate.

The current process of reconstituting a lyophilized oncology product is often performed either at the hospital or at a specialty compounding pharmacy by a licensed pharmacist. The use of a hood is often required to perform reconstitution steps to provide a sterile working environmental which can be cumbersome for a healthcare professional given the complexity of the procedure. In addition, this reconstitution process involves the use of multiple needles to withdraw/add sterile water for injection (WFI), saline and/or Intravenous Solution Stabilizer (IVSS) solutions. Typically, for relatively complex oncology products such as a Bi-specific T-cell Engager (BITE®) molecule (e.g. Blincyto®) prepared in an IV bag, a specified volume of WFI is added to reconstitute a lyophilized drug product contained in a vial via the use of a needle and syringe system. Then, the applicable volume of saline and IVSS solutions are added to an empty IV bag before the final reconstituted drug product is introduced. The overall process may involve relatively extensive manual labor time and steps. Often these steps include handling and/or use of needles, which may include inherent potential risks of needle-stick injuries.

As described in more detail below, the present disclosure sets forth systems and methods for drug delivery embodying advantageous alternatives to existing systems and methods, and that may address one or more of the challenges or needs mentioned herein, as well as provide other benefits and advantages.

### SUMMARY

In a first embodiment, a device for connecting tubes for a drug delivery system includes a connecting portion having a first tube channel, a second tube channel, a connecting heating element, and a carriage assembly. The first tube channel is adapted to receive a first tube, and the second tube channel is adapted to receive a second tube. The connecting heating element is positioned adjacent to and/or intersecting the first and second tube channels and is adapted to selectively heat at least a portion of the first and second tube. The carriage assembly moves the heated first and second tubes into end-to-end contact with each other to couple the first and second tubes together.

In some examples, the device includes a disconnection portion that includes a disconnecting heating element adapted to disconnect and seal the tube. The disconnecting heating element may be integrally formed with the connecting heating element. Further, in some examples, the device may include at least one blade adapted to segment the tube. In some approaches, a hinge member may be provided to selectively position the disconnecting portion. A locking mechanism may be used to retain the at least one blade in a closed position. In some examples, the connecting portion may further include a shield selectively movable to prevent access into the connecting portion.

In accordance with a second embodiment, a device for handling a drug product vial includes an elongated body having first and second ends, a dust-cap remover positioned at the first end of the elongated body, and a vial stabilizer positioned at the second end of the elongated body.

In accordance with a third embodiment, a device for fluidly connecting an IV line to a drug vial having a cap includes a cap clasp and a vial spike. The cap clasp includes at least two arms adapted to selectively couple with the cap of the vial. The vial spike is generally centrally located between the at least two arms and is configured to pierce a seal of the vial.

### BRIEF DESCRIPTION OF THE DRAWINGS

The above needs are at least partially met through provision of the systems and approaches for drug delivery device reconstitution described in the following detailed description, particularly when studied in conjunction with the drawings, wherein:

FIGS. 1a-1b illustrate an example tool for sealing and/or welding tube(s) for a flow path for a drug delivery system in accordance with various embodiments;

FIG. 2 illustrates an example drug delivery system in accordance with various embodiments;

FIG. 3 illustrates a perspective view of an example vial handling device for preparing a drug storage container in accordance with various embodiments;

FIG. 4 illustrates a top plan view of the example vial handling device of FIG. 3 in accordance with various embodiments;

FIG. 5a illustrates a top plan view of an alternative example vial handling device in accordance with various embodiments;

FIG. 5b illustrates a perspective view of the example vial handling device of FIG. 5a in accordance with various embodiments;

FIG. 6 illustrates an example vial connector device coupled with an example vial in accordance with various embodiments;

FIG. 7 illustrates the example vial connector device of FIG. 6 in accordance with various embodiments;

FIG. 8 illustrates an alternative example vial connector device coupled with an example vial in accordance with various embodiments;

FIG. 9 illustrates the example vial connector device of FIG. 8 in accordance with various embodiments; and

FIG. 10 illustrates an example drug delivery fluid path connection components in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

The present disclosure relates to a drug delivery system and, more particularly, to a drug delivery system including a pump and a fluid path for long-term, continuous, semi-continuous, and/or intravenous drug delivery. Under some conditions, a drug delivery process may last for an extended period of time (e.g., for one hour or longer) or may include continuous or semi-continuous delivery of a drug over an extended period of time (e.g., for several hours, days, weeks, or longer) or may include delivery via an intravenous connection to a patient. The present disclosure utilizes various features for potentially improved flexibility, usability, and patient convenience, while maintaining a relatively compact sized system that may be desirable or appropriate for extended, continuous, semi-continuous, and/or intravenous delivery.

For example, the present disclosure includes a tool that may be suitable for home use that is configured to enable patients and/or health care providers to selectively disconnect and/or connect from an IV infusion set. The tool may be used to repeatedly disconnect and/or connect the IV infusion set over the course of several days in a sterile manner without the use of several sterile connector/disconnectors integrated into the infusion time. Further, the present disclosure describes devices for handling drug product vials, vial connector devices, and fluid path connectors.

More specifically, a handheld tool is provided that integrates sealing and welding technologies into a single device. In some examples, when a patient wishes to disconnect from therapy, for example for hygienic purposes, the patient may activate a tube sealing element by selecting a sealing function. Once the desired activity is complete, the patient may activate a welding or connecting function.

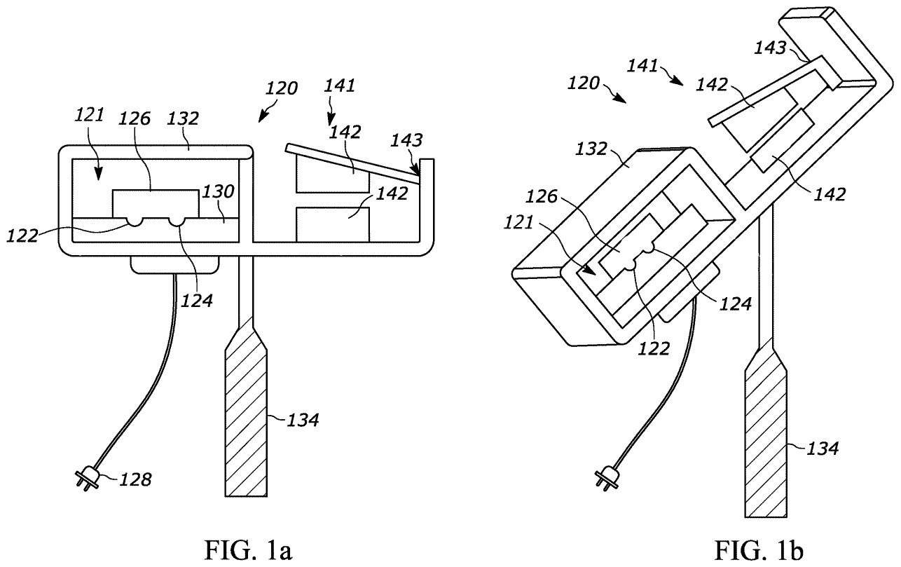

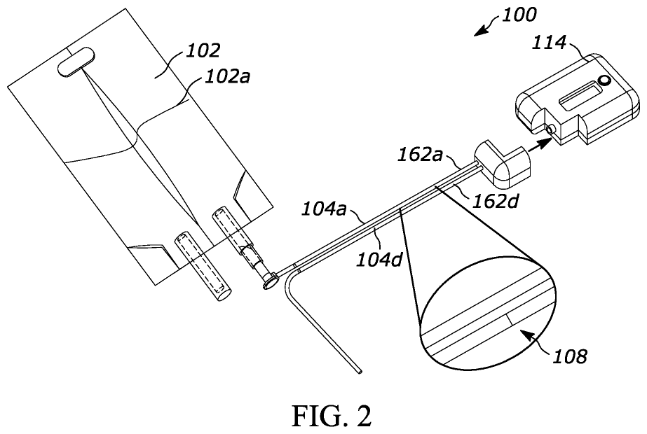

Turning to the figures, FIGS. 1a and 1b illustrate a portable, hand-held device 120 for coupling (e.g., welding or otherwise fusing) a number of tubes for a drug delivery system 100 (illustrated in FIG. 2). The device 120 includes a connecting/welding portion 121 and a disconnecting portion 141. The connecting/welding portion 121 includes a first tube channel 122 for receiving a first segment of a tube (e.g., a first portion of an output line 104d as illustrated in FIG. 2), a second tube channel 124 for receiving a second segment of a tube (e.g., a second portion of the output line 104d as illustrated in FIG. 2); and a heating element 126 positioned adjacent to and/or intersecting the first and second tube channels 122, 124 so as to selectively heat portions of the first and second portion of the output line 104d. For example, the output line 104d, which, in some examples may be constructed from a flexible polymeric material, may be heated to a point where a localized portion thereof becomes melted and/or molten. The device 120 may also include a handle 134 to grasp and manipulate the device 120.

The heating element 126 may be powered by a plug 128 or other power source/connector. In some examples, the heating element 126 may be a disposable, metal-based element that may increase in temperature via running a current generated by the plug 128 through opposing sides thereof. In other examples, the heating element 126 may be a ceramic-based element. Other examples are possible. In any of these examples, the heating element 126 may be heated to a specified temperature.

The device 120 shown in the figures may also include a carriage assembly 130 for moving and/or positioning the heated first and second tube channels 122, 124 such that the respective are likewise moved into end-to-end contact with each other to couple the first and second portions of the output line 104d together. The carriage assembly 130 may be spring loaded and/or have an alternative mechanical arrangement for moving the respective tubes together.

The device 120 may also include a safety shield 132. Further, the disconnecting portion 141 may include a sealing mechanism in the form of any number (e.g., a pair) of sealing blades 142 that are likewise workingly coupled with the heating element 126 or a different heating element. The sealing blades 142 may be used to discontinue or break a fluid path defined by a tube and optionally, to form a seal in the tube. An upper sealing blade 142 may be movably coupled via a hinge 143 to allow for selectively opening and closing the blades 142 relative to each other. The hinge 143 may be spring loaded such that it may be biased in either an open or closed configuration as desired. Further, in some examples, the hinge 143 may include a locking mechanism to maintain a closed and/or open configuration. Other examples are possible.

As illustrated in FIG. 2, an example drug delivery assembly 100 (or “system”) is provided that the hand-held device 120 may be used therewith. For example, the drug delivery assembly 100 includes a drug product container 102 for containing a drug product 102a (or medicament), an IV input line 104a, an IV output line 104d, each of which being in the form of tubing portions 162a, 162d leading to and from a pump 114, and a weld point 108 positioned along the tubing portion 162d. The tubing portion 162d is coupled with the user via any number of suitable approaches such as, for example, an IV needle or cannula. The weld point 108 is preferably fluid-tight, leak-free, and sterile. In some examples, the pump 114 may be worn by and/or otherwise coupled with the user.

In operation, to disconnect a portion of the system 100 from the user, the user may grasp the handle 134 and position the disconnecting portion 141 around the tubing portion 162d at a desired weld point 108. The device 120 may be heated using the plug 128, and the heated blades 142 may be moved towards each-other and retained in a closed configuration. The tubing portion 162d is then compressed and heated between the two blades 142 to a specified temperature which causes any liquid therewithin to be evacuated therefrom. Further, the heated blades 142 cause the tubing portion 162d to become melted and/or molten so that the tubing portion becomes segmented at the weld point 108. At this point, the heated blades 142 may be opened, and the tubing portion 162d will cool and solidify to create a sterile seal. The user may then perform the desired activity while being at least partially disconnected from the system 100. More specifically, the user may still be coupled with a portion of the tubing portion 162d, but will not be coupled with or connected to the drug container 102 or the pump 114.

Once the user has completed any desired daily activities, the user may position the segmented and sealed tubing portion 162d within the connecting portion 121 of the device 120. More specifically, one segment of the tubing portion 162d (e.g., the segment still connected with the user) is placed in the first tube channel 122, and the second segment of the tubing portion 162d (e.g., the segment connected with the pump 114) is placed in the second tube channel 124. The heating element 126 may then be engaged and positioned adjacent to the segments of the tubing portion 162d in the first and second tube channels 122, 124, and the carriage assembly 130 shifts the segments to align them (such as along a carriage) relative to each-other. In one example, the heating element 126 may cut or remove the sealed ends of the segments of the tubing portion 162d, and the carriage assembly 130 may then position the open ends of the tubing segments 162d adjacent to each other. The heating element 126 may then heated to be fused or welded together, and the heating element 126 may then be removed to allow the tubing portion 162d to cool and bond together. So configured, the device 120 is cost effective by not using an integrated sterile connector into each infusion line.

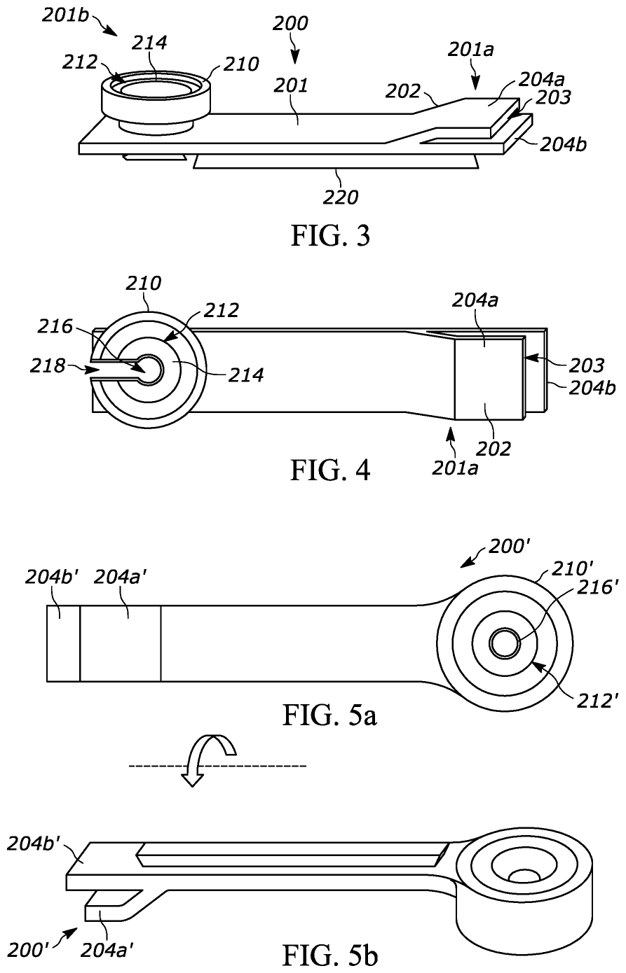

With reference to FIGS. 3 and 4, pharmacies often train staff to avoid/mitigate exposure to compounds through accidental needle-stick injury. Recently, many are mandating closed system transfer device (“CSTD”) usage for oncology therapies. For molecules that are incompatible with CSTDs, due to excess hold-up volume or with the material composition, a vial handling device 200 disclosed herein may reduce or prevent inadvertent needle-sticks. The vial handling device 200 may include a dust-cap remover 202, a vial stabilizer 210, and a storage mechanism 220. The vial handling device 200 includes an elongated body 201 having a first end 201a and a second end 201b. In the illustrated example, the dust-cap remover 202 is positioned at the first end 201a of the elongated body 201 and the vial stabilizer 210 is positioned at the second end 201b of the elongated body 201. Further, the storage mechanism 220 is positioned along the length of the elongated body 201.

The dust-cap remover 202 is in the form of a number of prongs 204a, 204b separated by a gap 203. The first prong 204a may have a curved, sharp, and/or tapered leading edge that may be inserted and/or wedged into a space between a vial and a dust cap to remove the dust cap from the vial. The vial may then be placed onto the vial stabilizer 210, which is in the form of a counter-sunk ledge that defines a first guide member 212 in which a vial may rest. In the illustrated example, the vial stabilizer 210 additionally includes a secondary guide member 214 to accommodate and retain smaller vials, and further includes a pass-through hole 216 and a pass-through channel 218 (FIG. 4). A needle may be inserted through the pass-through hole 216 to puncture the vial, and the vial handling device 200 may be removed from the punctured vial by passing the needle through the pass-through channel 218.

Advantageously, the elongated body 201 allows the vial handling device 200 to be used by a user while maintaining a safe, sterile distance from the vial by physically distancing the user from the vial, thereby potentially improving the safety of the handler. The device 200 is preferably able to be sterilized. Further, the storage mechanism 220, which in some examples may be a magnet, may assist with mounting the device 200 within a biosafety cabinet or workspace.

The illustrated example device 200′ of FIGS. 5a and 5b includes any number of the features of the device illustrated in FIGS. 3 and 4, but include an ergonomic elongated body 201′ and first and second ends 201a′, 201b′.

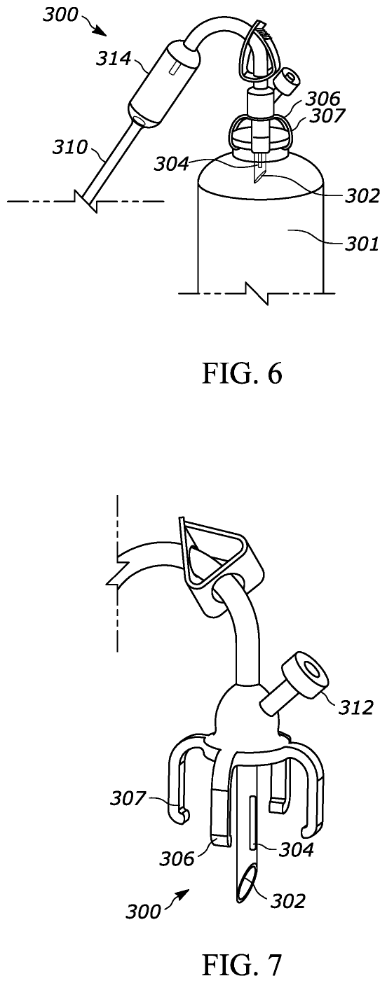

With reference to FIGS. 6-9, a vial connector device 300 is provided to permit a secure and stable attachment of a vial-specific IV-administration line. The device 300 may include a pre-sterilized vial-adapter-spike 302 which, in some examples, is manufactured with an attached IV-admin line 310 as a single unit having many of the standard features of IV-lines. The vial-adapter spike 302 may include a number of slots or channels 304 to decrease vial hold-up of the drug product. Further, the vial connector device 300 may include a clamping device 306 in the form of a bottle cap clamp to secure the spike 302 to the bottle 301 during administration. The clamping device 306 may include any number of flexible prongs 307 that may secure with an end of the vial 301.

The IV line 310 may have a small inner diameter for decreased hold-up volume, thus reducing priming time. The IV line 310 may be constructed from any number of suitable materials that are compatible with the drug product (e.g., non-PVC materials).

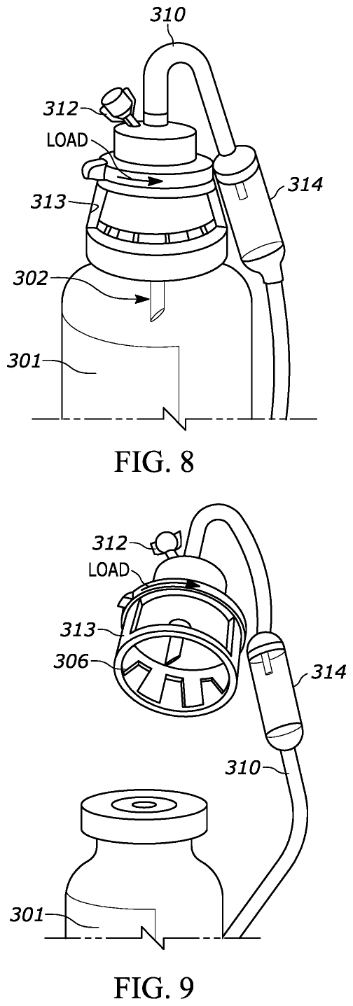

The connector device 300 may further include a drug-add port 312 to couple with a prefilled syringe (not illustrated) and may be in the form of a closed system or one way slide valve 313 (FIGS. 8 and 9). The one-way slide valve 313 may enable addition of the drug product, and may include a load position, an admixing process position, and an administration position. In the admixing position, the slide valve 313 may prevent the drug product from entering the IV line 310. In the administration position, after the drug product is mixed with the saline solution, the slide valve 313 may allow the solution to travel through the IV line 310 to be administered. Additionally, a custom drip chamber 314 is provided to limit hold-up volume.

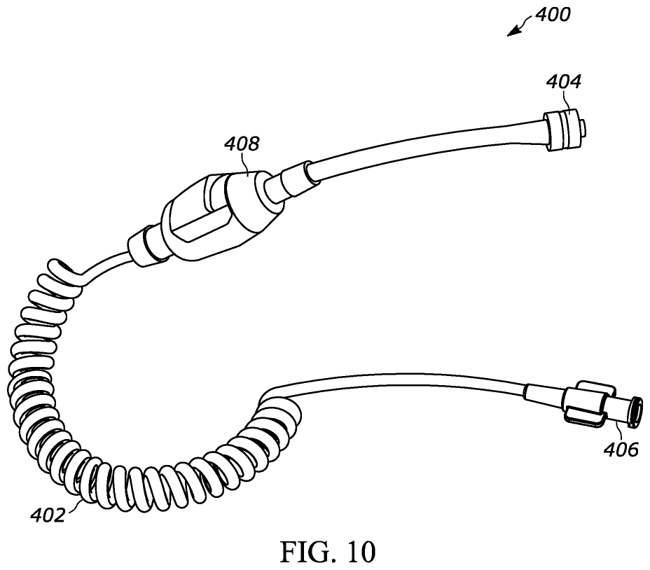

With reference to FIG. 10, a fluid path connector 400 is provided. In some examples, the fluid path connector 400 may be in the form of an IV Extension set, and may increase patient convenience and improve bag-change time as well as make a drug delivery system more portable/usable. For example, fluid path connector 400 may include a coiled line 402 and terminate in male and female luer connectors 404, 406. The IV extension may also have an aseptic coupler 408 integrated in line with the entire accessory provided in a sterile packaging.

So configured, the devices described herein seek to increase both safety and convenience for patients receiving continuous IV medication. Patients undergoing Continuous IV Therapies such as 5FU, Remodulin, Blincyto®, and other therapies may have challenges related to the IV line management going about typical daily activities. The inclusion of an aseptic coupler would improve their quality of life through simplification and reduced mental load related to these activities.

Currently many of these patients have a luer fitting that is either a port access line or a PICC line that enables direct access to the intravenous system. An IV extension set then connects either to a pump or directly to an IV bag to facilitate the delivery of therapy. In the case of short-term IV infusion, a patient might be connected by an HCP and sit in a chair or bed designed for IV infusions waiting for the infusion to take place. Unfortunately, because a patient is oftentimes connected 24/7 to the IV line, there are many seemingly simple tasks that may become complicated, such as getting dressed/undressed, taking a shower, and/or changing bags to avoid carrying a pump bag and purse or backpack.

If a patient has a home health or infusion nurse present then these tasks may be able to be made easier by pausing the pump, clamping the tubing, disconnecting the tubing, performing the task, cleaning the connections, reconnecting the tubing, unclamping the tubing, and restarting the infusion pump. Otherwise there may be an untangling task to figure out how to accomplish the task without disconnecting, or the potentially undesirable step of disconnecting without having a trained aseptic field and technique to prevent bloodstream infection (“BSI”). Inclusion of aseptic couplers on an integrated inline may be able to offer convenience without increasing the risk of BSI. The further inclusion of coiled lines or automatic retraction element can simplify allowing more mobility (needing longer lines) without increasing the risk of snagging (from sagging lines).

The drug delivery systems shown in the figures may include, generally, a drug product container containing a drug product; a diluent container containing a diluent; a saline container containing a saline solution; an IV stabilizing solution (“IVSS”) container containing an IVSS; and a fluid path assembly (IV line) configured to selectively, fluidly couple or de-couple various of the aforementioned components.

For example, the fluid path assembly may include a tubing manifold having a series of connection points for physically connecting the respective containers with each other. As a more specific example, the connection points may include quick-connect sterile connectors with respective sub-components that selectively mate with each other while maintaining sterility or another desirable cleanliness standard. For example, EldonJAmes SeriesLock™ quick disconnect fittings may be suitable. For example, the quick-connect sterile connectors may snap or twist or screw together; they may have sheathed or covered components that become unsheathed or uncovered upon connection; and/or they may have Luer Lock or modified Luer Lock configurations. As another example, the connectors may include one or more stake connectors for coupling one of the tube portions with an IV bag. The adaptors may be a closed system transfer device (“CSTD”) or a suitable vial adaptor that matingly fits with the drug product container, which may be a vial. Similarly, adapter may be a vial adaptor that matingly fits with the diluent container, which may also be a vial. Similarly, adaptor may be a vial adaptor that matingly fits with the IVSS container, which may be a vial. Any or all of the containers may be a vial with a standard septum that is pierced by a vial adapter or a vial stake; additionally or alternatively any or all of the containers may include a quick-connect sterile connector or other suitable connector. Alternatively, any or all of the containers may be a resilient container such as an IV bag or any other suitable container.

The saline container may be an IV bag, a vial, or any other suitable container. The saline container is coupled with the fluid path assembly via one or more ports. For example, IV spikes may pierce the ports to physically connect the saline container to the fluid path assembly. The IV bag is positioned in a packaging housing well for facilitate removal of air (by gravity). For example, the packaging housing bottom wall (which rests on a table or other surface) is preferably non-parallel with a bottom surface of the well for the IV bag so that air is urged towards the IV ports and can be more easily purged from the same.

In some examples, the IVSS may be provided as a percentage of an overall volume of solution. In these examples, suitable quantities of IVSS may range between approximately 2% and approximately 15% (e.g., between approximately 1 mL in a 50 mL container and approximately 25 mL in a larger, 270 mL container). The IVSS can also act as a pretreating surfactant or a buffering component that prevents adsorption of the drug onto the walls of the container. For example, due to the highly potent nature of some drugs being administered, if the container is not sufficiently and properly coated with the IVSS, it may lead to an undesirable risk of drug molecules adhering or adsorbing to the inner walls of the container. In the event of adsorption of the drug onto the delivery container walls, the dosage of the drug may be adversely impacted. In such a situation, it may be desirable to utilize the exemplary steps discussed in the prior paragraph.

In some examples, the IVSS may include polysorbate. In some examples, the IVSS formulation may include approximately 1.25 M lysine monohydrocholoride, 25 mM citric acid monohydrate, 0.1% (w/v) polysorbate 80, and has a pH of approximately 7.0. In other examples, the IVSS may include similar formulations, but also have a minimum of approximately 0.9% NaCl and approximately 0.001 to approximately 0.1% (w/v) polysorbate 80. It is appreciated that different BiTE®s require different final percentages of IVSS 54 in the delivery container. This percentage may vary between approximately 0.5% to approximately 12% of the final volume in the delivery container. Further, citrate may increase the risk of glass delamination if filled in glass vials. In the event that citrate is necessary for drug product stabilization (determined on a per-product basis), the delivery containers may be constructed from CZ or other plastic compositions. Other examples of ingredients for suitable IVSSs are possible. Suitable IVSS concentrations protect against protein-plastic interactions and/or surface adsorption, and more specifically, in the lower end of the concentration range where even minor losses may potentially change the effective dose. The below table illustrates example component concentrations for varying IVSS concentrations:

TABLE 1
Component Concentrations with Varying IVSS Concentrations (top column units are (V/v) % of IVSS

| | IVSS COMPONENTS (%) | | | | | | | |
|---|---|---|---|---|---|---|---|---|
| | 0.5 | 1.0 | 2.0 | 4.0 | 6.0 | 8.0 | 10.0 | 12.0 |
| Lysine | 0.00625 | 0.0125 | 0.025 | 0.05 | 0.075 | 0.1 | 0.125 | 0.15 |
| monohydrochloride (M) | | | | | | | | |
| Citrate Monohydrate (M) | 0.000125 | 0.00025 | 0.0005 | 0.001 | 0.0015 | 0.002 | 0.0025 | 0.003 |
| Polysorbate 80 (% w/v) | 0.0005 | 0.001 | 0.002 | 0.004 | 0.006 | 0.008 | 0.01 | 0.012 |

By providing the components in containers that are selectively connectable, it may be no longer necessary to prepare a needle and syringe assembly to inject one component into another container, to ensure that this prepared needle and syringe assembly is sterilized, and/or to ensure a correct volume or amounts of components are added together.

Some conventional systems may provide delivery containers having saline solution overfill, where more saline solution is provided in the delivery container than what is needed for dosage. In these systems, it may be necessary to remove a volume of the saline solution prior to preparing the drug dosage, which may require preparing a sterile withdrawal tool (e.g., a needle and syringe assembly) and carefully extracting an accurate amount of saline solution. Conversely, the disclosed systems may additionally eliminate this process, as the containers are prefilled with the required quantity of components. Additionally, the risk of a needle sticking due to the transfer of the components may also be reduced or mitigated.

Additionally, many or all of the above described steps may be automated or semi-automated or reduced in time/scope, thereby potentially saving time and effort for the persons preparing and/or using the drug.

As discussed above, the drug product container contains a predetermined quantity of drug product or active pharmaceutical ingredient (“API”) (e.g., between approximately 2 mcg and approximately 100 mcg), depending on the BiTE® and container size, which, in the illustrated example, is in powdered form (i.e., lyophilized) requiring reconstitution. In other examples, the drug product may be in liquid form and may not require reconstitution. Nonetheless, the system includes an accurate quantity of drug product, and thus does not require the need to add additional quantities thereto in a sterile environment. In some examples, the API may be in the form of a half-life extended (“HLE”) BiTE® and/or an IV-admin monoclonal antibody (“mAbs) as desired. These HLE BiTE®s include an antibody Fc region that advantageously provides different drug properties such as longer and extended half-lives. Accordingly, such APIs may be preferred due to their ability to maintain protective levels in the patient for relatively longer periods of time. Nonetheless, in other examples, the API may be in the form of a canonical-BITE® that is to be administered in a professional healthcare environment.

In some embodiments, the drug delivery system may have an integrated reconstitution subsystem onboard to dilute a lyophilized drug into a liquid form. In certain such embodiments, a diluent reservoir may be included for storing a diluent solution and a lyophilized reservoir may be included storing a lyophilized compound separate from the diluent solution. Furthermore, a fluid drive mechanism may be included for mixing the diluent solution in the diluent reservoir with the lyophilized compound in the lyophilized reservoir. In some embodiments, the fluid drive mechanism may transfer the diluent solution from the diluent reservoir into the lyophilized reservoir and/or provide any circulation and/or agitation needed to achieve full reconstitution. In some embodiments, an additional final reconstituted drug reservoir may be included and serve as a delivery reservoir from which the reconstituted drug is discharged into the patient; whereas, in other embodiments, the lyophilized reservoir may serve as the delivery reservoir. While the reconstitution subsystem may be physically integrated into the drug delivery system in certain embodiments, in other embodiments the reconstitution subsystem may constitute a separate unit which is in fluid communication with the drug delivery system. Having a separate unit may simplify the reconstitution process for healthcare providers in certain cases.

The drug product container may be in the form of an IV bag, a vial, a prefilled syringe, or similar container that includes a reconstitution container body defining an inner volume. The inner volume may be sterile. In some approaches, the reconstitution container adapter may also be a CSTD (or, in examples where the prefilled reconstitution container is in the form of a syringe, the container adapter may be a needle) that mates, engages, and/or couples to the vial adapter. Additionally or alternatively, the drug product can be bulk lyophilized and filled into a cartridge or container that is typically used to administer with an IV pump. If needed the dehydrated forms of IVSS, NaCl, and any other components needed for the final administered solution can be bulk lyo'ed and filled into the cassette for long term storage.

The prefilled diluent container contains a predetermined quantity of diluent (e.g., preservative-free water for injection or “WFI”) (e.g., between approximately 0.5 mL and approximately 10 mL) to be added to the prefilled drug product container for reconstitution of the drug product. In some examples, a benzyl alcohol preserved (or any other preservative) WFI may be used.

As previously noted, in some examples, the prefilled drug product container may be in the form of a prefilled syringe that contains the drug product. In these examples the drug product may be in the form of a liquid BiTE® formulation used in conjunction with a monoclonal antibody (mAb), In these examples, the drug product may be directly added to the delivery container without the use of a vial adapter system (such as the above-mentioned CSTDs) where more traditional needle-syringe injection/delivery into the container is preferred, which may advantageously simplify and/or improve supply chain and manufacturing control, and may further allow for more compact commercial packaging that takes up less space in storage systems at healthcare facilities. In these examples, the prefilled drug product vial may or may not need to be reconstituted prior to transferring the drug product to the delivery container.

The system may be distributed and/or sold as a common kit packaging, but other suitable distribution/packaging is suitable. The drug product may be in the form of a half-life extended bispecific T cell engager (BITE®), but other drug products are suitable. The diluent may include WFI, but other diluents may be suitable. The containers may be pliable bags, such as IV bags, but other containers may be suitable. In some examples, one or more of the containers is in the form of an IV drip bag constructed from a plastic or other material, e.g., 250 mL 0.9% Sodium Chloride IV bag constructed of a suitable material such as polyolefin, non-DEHP (diethylhexl phthalate), PVC, polyurethane, or EVA (ethylene vinyl acetate) and can be filled to a volume of approximately 270 mL to account for potential moisture loss over long-term storage.

During some or all of the above steps, the contents of a container may then be gently stirred, swirled, and/or inverted to mix the ingredients, thereby forming a desired mixture. Similarly, the mixtures may be visually inspected for imperfections and/or to ensure adequate mixing has occurred.

The system may be used to provide intravenous, subcutaneous, intra-arterial, intramuscular, and/or epidural delivery approaches. By using the system, patient anxiety and or confusion may be reduced due to reduced preparation complexity and wait times caused by the drug preparation process.

In some examples, the prefilled delivery container is in the form of an IV drip bag constructed from a plastic or other material, e.g., 250 mL 0.9% Sodium Chloride IV bag constructed of a suitable material such as polyolefin, non-DEHP (diethylhexl phthalate), PVC, polyurethane, or EVA (ethylene vinyl acetate) and can be filled to a volume of approximately 270 mL to account for potential moisture loss over long-term storage. Other examples of suitable delivery containers are possible such as, for example, a glass bottle or container. Example suitable prefilled delivery containers are described in U.S. Appln. No. 62/804,447, filed on Feb. 12, 2019 and U.S. Appln. No. 62/877,286 filed on Jul. 22, 2019, the contents of each of which are incorporated by reference in their entirety.

At least one of the delivery container adapters may be a closed system transfer device (“CSTD”) that allows for transfer of the drug and/or fluids into the container body. Example CSTD devices may include the OnGuard CSTD provided by B. Braun Medical Inc, BD PhaSeal CSTD components, Equashield CSTD, Codon CSTD, and the like. Further, non-closed system transfer devices may be used such as West Pharmaceuticals vial and bag adapters. Other examples are possible. The prefilled delivery container may include any number of delivery container adapters having different specifications (e.g., port sizes) to accommodate the use of different drug product vials.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4ß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Solids™ (eculizumab); pexelizumab (anti-05 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP Ilb/Ilia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-a4integrin mAb); Valortim® (MDX-1303, anti-B. anthracis protective antigen mAb); ABthrax™ Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCG8 mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFß mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™ or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BITE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A device for connecting tubes for a drug delivery system, comprising:
a connecting portion having a first tube channel adapted to receive a first tube, a second tube channel adapted to receive a second tube, and a connecting heating element positioned adjacent to and/or intersecting the first and second tube channels and adapted to selectively heat at least a portion of the first tube and the second tube, and a carriage assembly adapted to move the heated first and second tubes into end-to-end contact with each other to couple the first and second tubes together.

2. The device of claim 1, further including a disconnecting portion including a disconnecting heating element adapted to discontinue and seal the tube.

3. The device of claim 2, wherein the disconnecting heating element is integrally formed with the connecting heating element.

4. The device of claim 2, further comprising at least one blade adapted to segment the tube.

5. The device of claim 2, further comprising a hinge member adapted to selectively position the disconnecting portion.

6. The device of claim 5, further comprising a locking mechanism adapted to retain the at least one blade in a closed position.

7. The device of claim 1, wherein the connecting portion further includes a shield selectively movable to prevent access into the connecting portion.

8. A drug delivery assembly for delivering a drug to a user, the drug delivery assembly comprising:
a container for containing a drug;
a pump operably coupled with the container for dispensing the drug to a user;
an output line operably coupled to the pump and the user to deliver the drug to the user; and
the device of claim 1.

9. A device for handling a drug product vial, comprising:
an elongated body having a first end and a second end;
a dust-cap remover positioned at the first end of the elongated body; and
a vial stabilizer positioned at the second end of the elongated body.

10. The device of claim 9, wherein the dust-cap remover includes at least one prong adapted to wedge under a dust cap covering from the drug product vial.

11. The device of claim 9, wherein the vial stabilizer includes a first guide member comprising a counter-sunk ledge.

12. The device of claim 11, wherein the vial stabilizer further includes at least one of a pass-through hole or a pass-through channel.

13. The device of claim 9, further comprising a securing mechanism positioned along the elongated body to secure the device to a storage container.

14. A device for fluidly connecting an IV line to a drug vial having a cap, comprising:
a cap clasp having at least two arms adapted to selectively couple with the cap of the vial; and
a vial spike generally centrally located between the at least two arms and configured to pierce a seal of the vial.

15. The device of claim 14, wherein the at least two arms are flexibly positionable around the cap of the vial.

16. The device of claim 14, wherein the vial spike includes at least one slot formed thereon.

17. A drug fluid path coupler comprising:
a tube having a coiled line portion, a female end, and a male end; and
an aseptic coupler positioned along the tube, between the female end and the male end, and configured to permit a sterile disconnection and connection of the tube.

18. The drug fluid path coupler of claim 17, further comprising a luer connector at the male end and/or the female end.

19. The drug fluid path coupler of claim 17, further comprising a sterile packaging in which the entire drug fluid path coupler.

20. The drug fluid path coupler of claim 17, wherein the aseptic coupler is integrated in line with the tube.
